(19) United States
(12) Patent Application Publication
(10) Pub. No.: US 2026/0148817 A1
(43) Pub. Date: May 28, 2026

(54) CAPTURE AND USE OF RESULTS FOR DIAGNOSTIC ASSAYS VIA POINT-OF-COLLECTION DEVICES USING MOBILE DEVICES

(71) Applicant: IN VITRO DIAGNOSTIC SOLUTIONS, Mount Laurel, NJ (US)

(72) Inventors: Robert HARPER, Mount Laurel, NJ (US); George TEMENG, Mount Laurel, NJ (US); Marc EBERLE, Mount Laurel, NJ (US); Matthew DiCERBO, Mount Laurel, NJ (US)

(21) Appl. No.: 19/121,201

(22) PCT Filed: Oct. 19, 2023
(86) PCT No.: PCT/US2023/077275

§ 371 Date: Apr. 15, 2025

Related U.S. Application Data
(60) Provisional application No. 63/417,856, filed on Oct. 20, 2022.

(51) Int. Cl. G16H 10/65; G16H 10/40; G16H 50/20

(52) U.S. Cl. CPC G16H 10/65; G16H 10/40; G16H 50/20

(57) ABSTRACT

Methods and devices are provided for capturing and using quantitative and qualitative results from point-of-collection devices for different diagnostic assays on a mobile device application. In one nonlimiting embodiment, these methods and devices are used in the diagnosis and/or monitoring of phenylketonuria (PKU).

This patent application claims the benefit of priority from U.S. Provisional Application Ser. No. 63/417,856, filed Oct. 20, 2022, teachings of which are herein incorporated by reference in their entirety.

### FIELD OF THE INVENTION

The present invention relates to computer-implemented methods and devices for capturing and using quantitative and qualitative results from point-of-collection devices for different diagnostic assays through mobile devices. In one nonlimiting embodiment, the methods and devices are used to diagnose phenylketonuria (PKU) and/or to monitor L-phenylalanine (Phe) in subjects with PKU.

### BACKGROUND OF THE INVENTION

Point-of-care or point-of-collection (POC) testing, also referred to as near patient, bedside, or extra laboratory testing, is not new. Many early diagnostic tests were first done at the bedside. More recently, however, analytical systems have been developed that enable a wider range of diagnostic tests which can be performed quickly and easily without the need for sophisticated laboratory equipment.

A key objective of POC testing is to generate a result quickly so that appropriate treatment can be implemented, leading to an improved clinical and/or economic outcome.

POC tests can be performed in a wide variety of locations including, but not limited to, your home, a health care practitioner's office, the emergency department, an infectious disease containment unit, ambulances, at an accident scene, in the military, in the radiology department, on a cruise ship, or even on the space shuttle. Further, a wide variety of people can perform POC tests, including laboratory professionals, emergency first responders, radiologists, doctors, nurses, physician assistants, or other health care practitioners. They may even be done by the patient themselves, sometimes called “self-tests” or “home tests.”

The most common POC tests are blood glucose monitoring and home pregnancy tests. Other common tests are for hemoglobin, fecal occult blood, rapid strep, as well as prothrombin time/international normalized ratio (PT/INR) for people on the anticoagulant warfarin.

Devices for POC tests come in an array of forms. They may use basic dipsticks as with urinalysis, handheld devices like glucose meters, or sophisticated molecular analyzers to detect infectious diseases. Diagnostic reagent strips, with 1 or more reagent pads adhered to a plastic handle, are one of the most common testing technologies in routine clinical use.

A colorimetric assay, referred to as “PKU Now”, useful at the POC, at home, in the hospital, or at a clinician's office to measure L-phenylalanine (Phe) and for the diagnosis of phenylketonuria (PKU) is disclosed in U.S. Pat. No. 10,830,765.

However, many advances in diagnostic technologies are still complex, expensive and limited to centralized healthcare facilities or research laboratories, thus hindering use of diagnostics in resource limited settings and primary care.

With the escalating use of smart phones, there has been a rapid growing trend for adapting them into sensing and diagnostic needs related to medical health care. A review of smartphone-based diagnostic technologies is provided by Hernadez-Neuta et al. (Journal of Internal Medicine 2018 285:19-39).

A Phone Screen Testing (POST) method which detects SARS-COV-2-positive individuals by RT-PCR testing of smartphone screen swab samples has been disclosed to exhibit high sensitivity (81-100%) compared to nasopharyngeal RT-PCR SARS-COV-2 test results from individuals with a high viral load (Young et al. eLife 2021 10: e70333 pages 1-13).

As medical care evolves to become more consumer-focused, POC testing will continue to be an important way to perform medical testing. To receive the highest quality care using POC tests, it is important that the POC tests are part of a testing continuum that includes centralized clinical laboratories and a team of health care practitioners.

### SUMMARY OF INVENTION

An aspect of this disclosure relates to a computer-implemented method for capturing and using quantitative and qualitative results from POC devices for different diagnostic assays on a mobile device application. The method comprises connecting a mobile device to a meter of a POC diagnostic device. A unique code associated with a selected diagnostic assay or test strip lot is then scanned or imaged by the mobile device. Data from the meter of the POC diagnostic device associated with the selected diagnostic assay is then transmitted to the application on the mobile device and a diagnostic test result is calculated from the transmitted data by the mobile device application. Diagnostic test results calculated via the method may be immediately accessible and displayed on the mobile device by the mobile device application and/or stored for future viewing and/or dissemination remotely.

In one nonlimiting embodiment, this computer-implemented method is used diagnose PKU and/or to monitor Phe levels in subjects with PKU.

Another aspect of this disclosure relates to devices for capturing and using quantitative and qualitative results from POC devices for different diagnostic assays through mobile devices. The device comprises a means for accessing a computer-implemented method for capturing and using quantitative and qualitative results from POC devices for different diagnostic assays on a mobile device application and a POC diagnostic device which can connect to said mobile device. In one nonlimiting embodiment, the POC diagnostic device comprises a meter and test strips for selected diagnostic assays.

In one nonlimiting embodiment, this device is used to diagnose PKU and/or to monitor Phe levels in subjects with PKU.

Yet another aspect of this disclosure relates to a method of testing and/or diagnosing a patient with this method and/or device.

In one nonlimiting embodiment, this testing and/or diagnostic method is used to diagnose PKU and/or to monitor Phe levels in subjects with PKU.

### BRIEF DESCRIPTION OF THE FIGURES

FIG. 1 is a diagram depicting steps involved in a nonlimiting embodiment of the present invention in capturing and using quantitative diagnostic assay results from a POC device meter via a mobile device application.

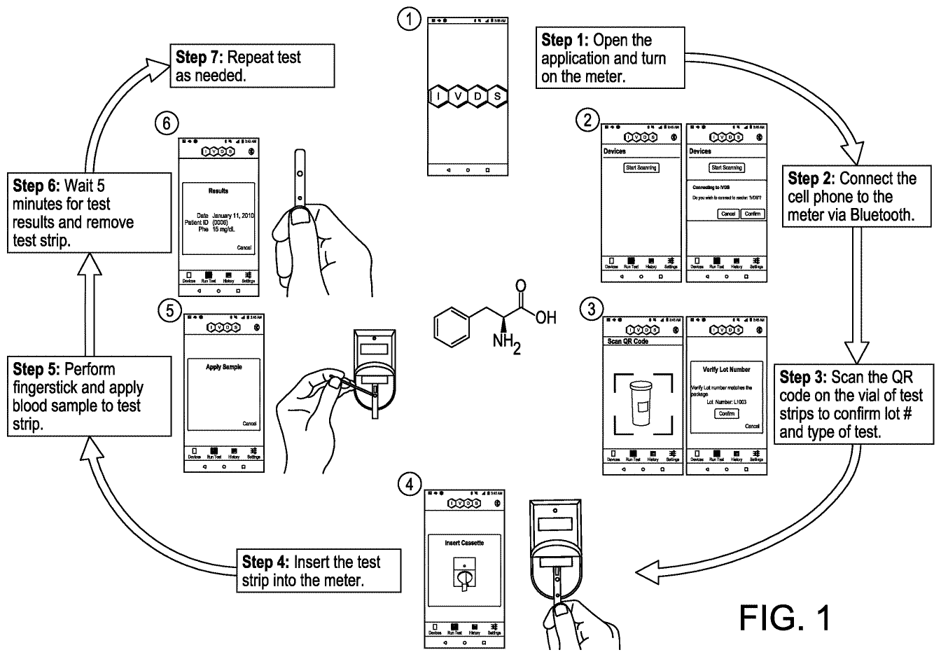

FIG. 2 shows a nonlimiting embodiment of a kit for use with this quantitative diagnostic assay inclusive of a POC device meter, test strips with a QR code, a holder for the test strip and a blood draw kit as well as the mobile device application loaded to a mobile phone.

FIG. 3 is a diagram of a nonlimiting example of a POC device meter.

FIGS. 4A and 4B show a front view (FIG. 4A) and a back view (FIG. 4B) of the POC device meter of FIG. 3 with labels to the power LED 1, power switch 2, cassette guide 3, LEDs 4, test strip insertion point 5, rubber feet 6, and battery door 7.

FIGS. 5A and 5B show a top view (FIG. 5A) and bottom view of the test strip with labels to the alignment indicator (arrow) 8, sample well 9, strip handle 10, and read zone 11.

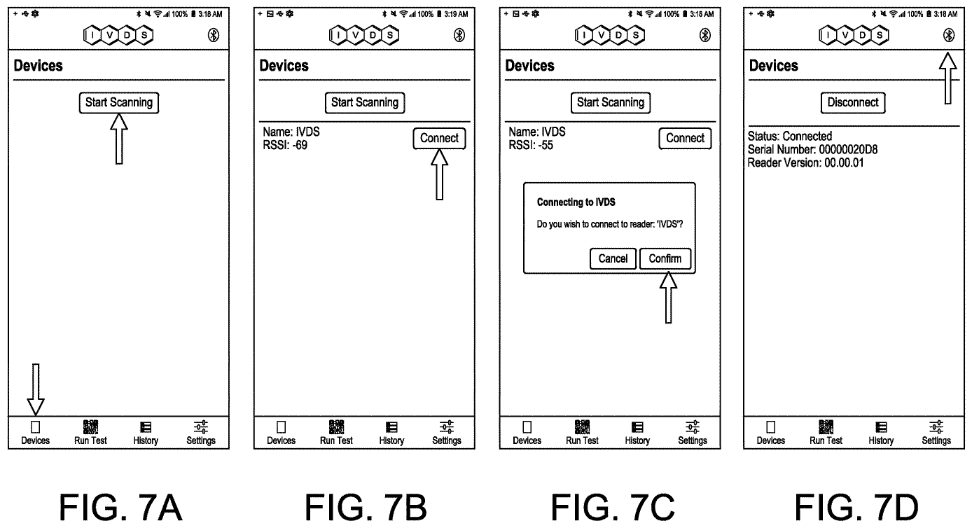

FIG. 6 is a screenshot of a mobile device with an icon for the mobile device application.

FIGS. 7A-7K are screenshots of a nonlimiting embodiment of the mobile device application. In this nonlimiting embodiment, FIG. 7A shows scanning for devices via Bluetooth on the devices tab, FIG. 7B shows connecting to an available meter, FIG. 7C shows confirmation of the connection, FIG. 7D shows connected device information, FIG. 7E shows starting of testing by scanning the code on the test strip vial, FIG. 7F shows verification of the test strip lot number and expiration date; FIG. 7G shows input of a patient identification, FIG. 7H shows prompting of the user to insert a test strip, FIG. 7I shows prompting of the user to start the test, FIG. 7J shows a potential error message during testing, FIG. 7K shows a prompt to apply the sample, and FIG. 7L shows indication by the mobile application that testing is in progress.

FIG. 8 shows screenshots of the mobile device application during control testing.

FIG. 9 is a curve generated from the percent reflectance values obtained from blood samples spiked with phenylalanine.

FIG. 10 shows agreement between gravimetric measurements of Phe concentration and predicted Phe concentration using the disclosed meter.

### DETAILED DESCRIPTION OF THE INVENTION

This disclosure relates to computer-implemented methods and devices for capturing and using quantitative and qualitative results from POC devices for different diagnostic assays on a mobile device application.

In the computer-implemented method of this disclosure, a meter of a POC diagnostic device is connected to a mobile device.

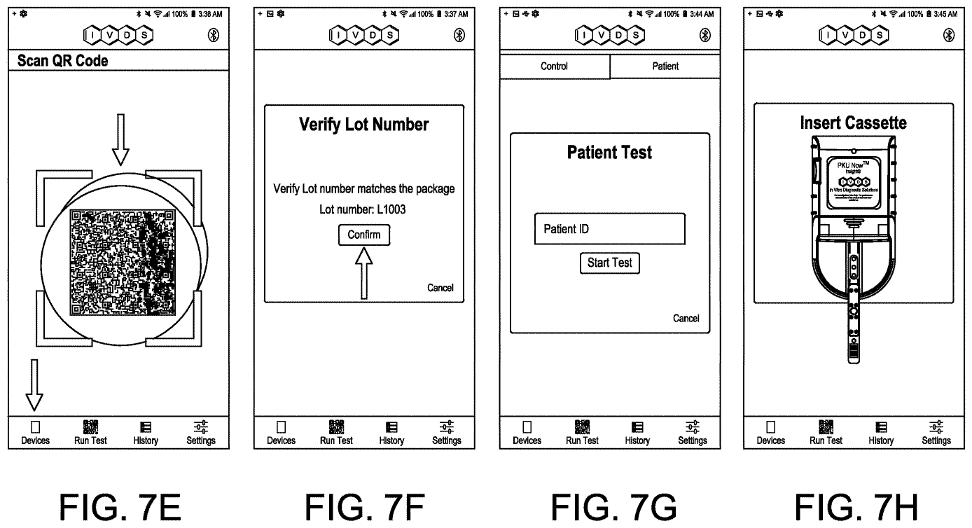

By “mobile device” for use in this method it is meant any handheld computer or smartphone. Nonlimiting examples of mobile devices include tablets, e-readers, smartphones, PDAs, portable music players, smartwatches, and fitness trackers with smart capabilities. A smartphone with an icon for the mobile device application of this invention is depicted in FIG. 6.

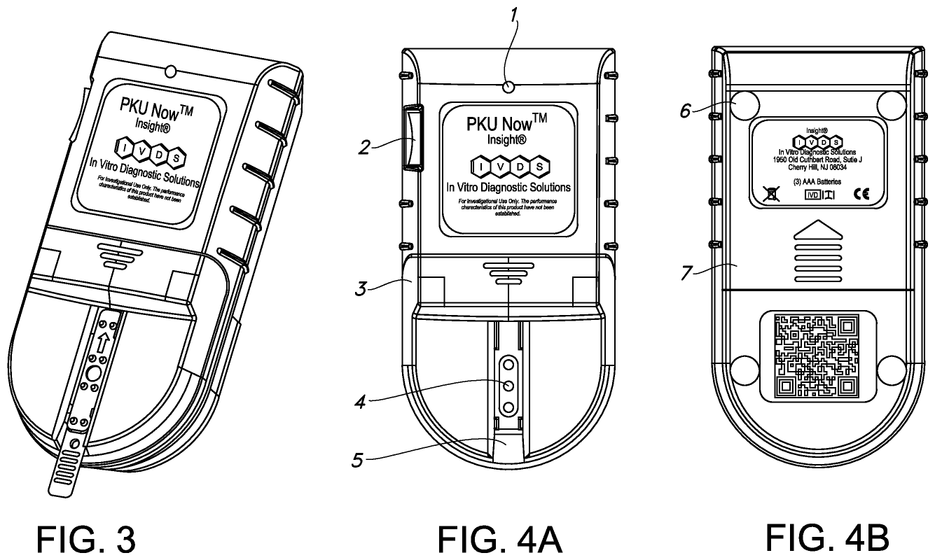

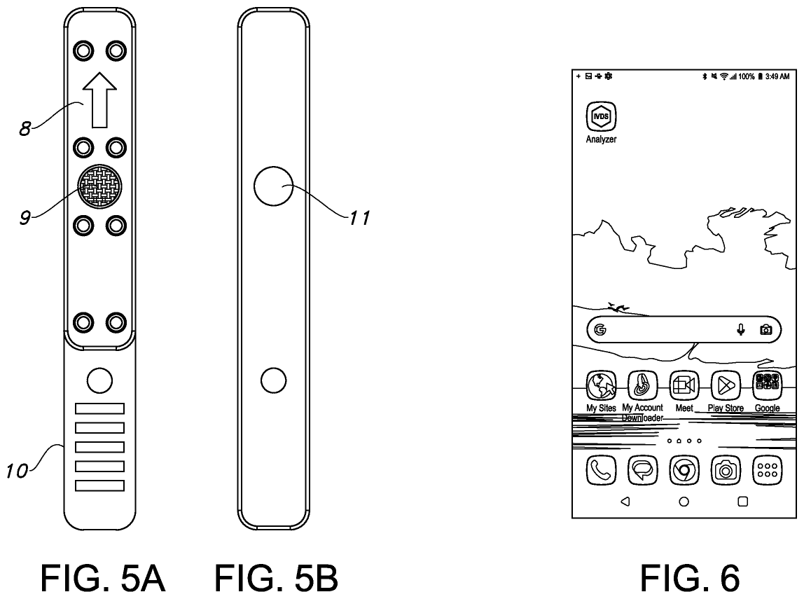

Nonlimiting examples of POC diagnostic meter devices which can be connected to a mobile device in accordance with this disclosure include devices by Polymer Technology Systems, CardioChek, A1C Now, Accu Answer, Zealson, Mission, Nesco, True Metrix, MOBI, Digital thermometer, Ultra Trak, Easy Home, Free Style and Hemocue. A nonlimiting example of a POC diagnostic meter device for use in the methods disclosed here is depicted in FIGS. 3-4.

In one nonlimiting embodiment, the mobile device is connected to the meter via wireless connectivity. In one nonlimiting embodiment, the wireless connectivity is a short-range wireless technology such as Bluetooth.

In one nonlimiting embodiment, the mobile device is connected to the meter via near field communication (NFC).

In one nonlimiting embodiment, the mobile device is connected to the meter radio frequency identification (RFID).

A unique code associated with a selected diagnostic assay or test strip lot is then scanned or imaged via the mobile device. Nonlimiting examples of unique codes include, a QR Code (QR Code is registered trademark of DENSO WAVE INCORPORATED) or a bar code.

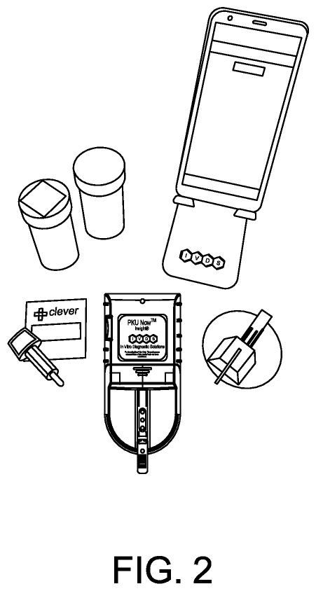

In one nonlimiting embodiment, the unique code is placed on the vial of diagnostic test strips such as depicted in FIG. 2.

The unique code is associated with a selected diagnostic assay and one or more parameters selected from calibration curves, with transformations including, but not limited to exponential, linear, logarithmic, polynomial, power, moving average, log and K/S transformation, endpoint determination, temperature correction algorithms, lot number, lot expiration, timing sequences, event markers and/or quality metrics in an application associated with the selected diagnostic assay which are stored in a mobile device application on the mobile device. The mobile device application may comprise parameters for various different diagnostic assays including, but in no way limited to, lactate, lactate dehydrogenase, ALT, AST, GGT, phenylalanine, glucose-6-phosphate dehydrogenase, adenosine deaminase, oxygen carrying capacity of lipids, lipids, cholesterol, glucose, uric acid, methionine, tuberculosis, free fatty acids, acyl-CoA, ATP, pyruvate, maltose, glutamate, phosphate pyrophosphate, creatine, creatinine, sarcosine, formaldehyde, glycine, formate, urea, choline, Co2, O2, amylase, maltose, tryptophan, beta-hydroxy butyrate, ketones, aldehydes, phospholipids, carbohydrates, sialic acid, nucleic acids, hemoglobin, hemoglobin A1C, urea, and nitrogen.

In one nonlimiting embodiment, the diagnostic test strip used in these methods and devices is comprised of a plurality of superimposed layers which spread, separate and detect a selected component in a biological sample which is then read by a POC diagnostic meter device.

Data from the meter of the POC diagnostic device are then transmitted to the mobile application on the mobile device. Data is generated from a diagnostic test strip contacted with a patient sample which is then read by the meter. Data transmitted may comprise photometric, amperometric, conductimetric and/or potentiometric data.

In one nonlimiting embodiment, data from the meter are transmitted to the mobile device via wireless connectivity.

In one nonlimiting embodiment, data from the meter are transmitted to the mobile device via near field communication (NFC).

In one nonlimiting embodiment, data from the meter are transmitted to the mobile device via radio frequency identification (RFID).

A diagnostic test result is then calculated by the mobile device application from the transmitted data. The diagnostic test result may be calculated via the mobile device application from one or more parameters selected from calibration curves, transformation, endpoint determination, temperature correction algorithms, lot number, lot expiration, timing sequences, event makers and/or quality metrics in the mobile device application. Diagnostic test results may be qualitative or quantitative.

In some embodiments, the computer-implemented method further comprises a means for inputting patient identification associated with the diagnostic test result. Such means may provide for manual entry of patient information into the mobile device application.

Alternatively, patient information may be associated with a patient specific code imaged by the mobile device.

Diagnostic test results may be immediately accessible and displayed on the mobile device by the mobile device application and/or they may be stored for future viewing and/or dissemination remotely. In some embodiments, the diagnostic test result is transmitted via the mobile device application to a health care provider or database such as, but not limited to, a cloud-based database to which a health care provider has access.

Also provided by this disclosure are devices for capturing and using quantitative and qualitative results from POC devices for different diagnostic assays through mobile devices. Such devices comprise a means for accessing the computer-implemented method for mobile devices disclosed herein and a POC diagnostic device which can connect to the mobile device. In one nonlimiting embodiment, the POC diagnostic device comprises a meter and test strips for one or more selected diagnostic assays.

Also provided by this disclosure are methods for calculating a diagnostic test result in a patient via the methods and devices disclosure herein. In these methods, a biological sample obtained from a patient is applied to a diagnostic test strip. Nonlimiting examples of biological samples for this method include blood, urine, sputum, and throat and/or nasal swabs. A diagnostic result indicated by the test strip is then calculated via the method and/or device disclosed herein.

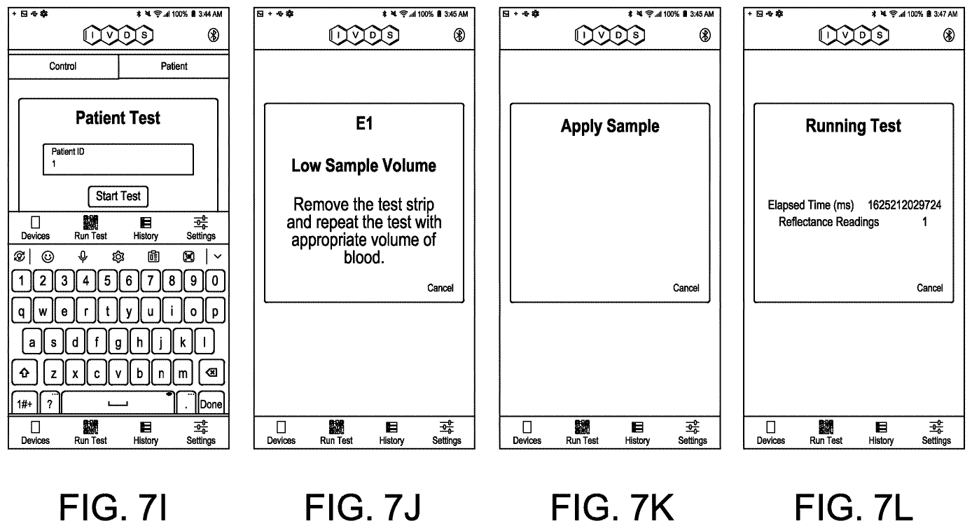

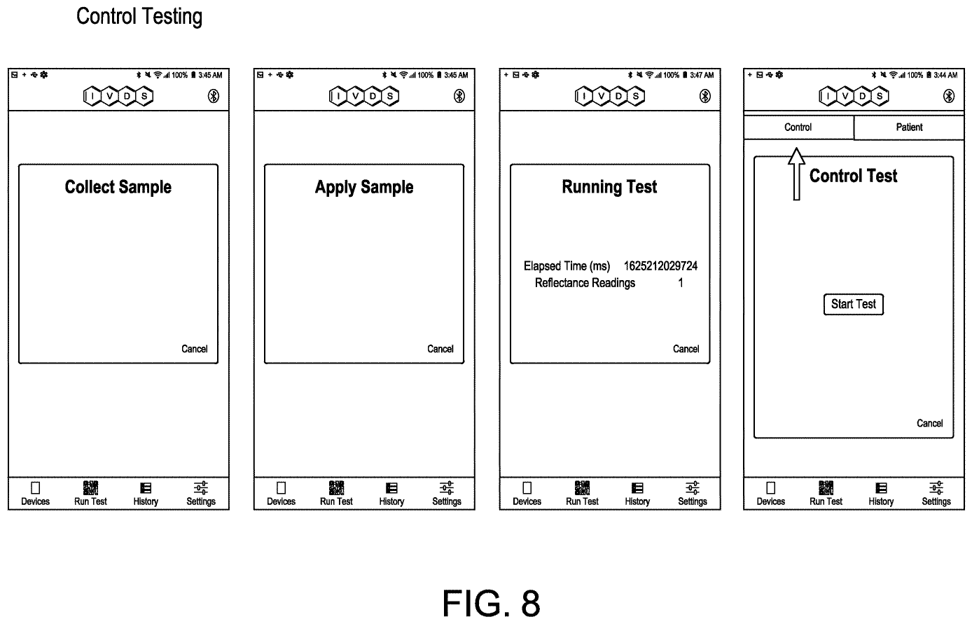

Nonlimiting examples of screenshots showing the mobile device application in use are depicted in FIGS. 7A-7L. FIG. 8 provides nonlimiting examples of screenshots showing the mobile device during control testing. The following Table 1 shows some of the error codes which may be generated during use of method and device of this disclosure.

TABLE 1

| ERROR | TEXT | HOW TO CORRECT |
|---|---|---|
| E-1 | Low sample | Remove the test strip and |
| | volume | repeat the test with |
| | | appropriate volume of |
| | | blood. |
| E-2 | Strip not inserted | Remove the test strip and |
| | correctly | insert the strip correctly. |
| E-3 | The test strip has | Remove the used strip and |
| | been used | perform the test with an |
| | | unused test strip. |
| E-4 | Incorrect timing of | The time it took to apply |
| | procedure | sample was too long. |
| | (specimen | Remove test strip and |
| | application) | repeat the test process. |
| E-5 | Strip or test error | Repeat the test with a new |
| | | test strip. |
| E-6 | Too hot/cold to | Remove the test strip and |
| | perform accurate | move the meter to an |
| | test | appropriate environment |
| | | for 20 minutes. |
| E-7 | Meter software or | Turn the meter off and on. |
| | hardware | If the error persists, reach |
| | malfunction | out to customer service. |
| E-8 | Test strip has been | Remove test strip and |
| | manipulated | repeat test with another |
| | | strip. |
| E-9 | Wrong test strip | Insert the correct test strip |
| | for phenylalanine | for the PKU Now |
| | test | |
| E-10 | Expired test strip | Use a test strip that is not |
| | | expired. |

Phenylketonuria (commonly known as PKU) is an inherited disorder that causes a toxic buildup of the amino acid L-phenylalanine (Phe) in the blood. PKU is the most common disorder of amino acid metabolism and occurs in 1 out of every 8,000 newborns globally. Most cases of PKU are detected by newborn screening in developed countries shortly after birth, and treatment is typically started promptly. Once newborns are diagnosed with PKU, L-phenylalanine levels must be monitored frequently to ensure they fall within acceptable levels (2-6 mg/dL). Without proper monitoring and treatment, affected infants can develop permanent intellectual disabilities. Seizures, delayed development, behavioral problems, and psychiatric disorders are also common side effects. The testing guidelines are as follows: infants<4 weeks old should be tested 1-2 times per week; infants 4-12 weeks old should be tested 1 time per week; children 1-2 years and older should be tested 2-4 times per month; women who are pregnant should be tested 1-2 times per week; and patients who are ill should be tested 1 time per week as directed by their clinician.

Currently, there is no FDA-approved POC device commercially available for measuring Phe levels in whole blood.

The computer-implemented method and device disclosed herein was demonstrated to be useful in monitoring Phe levels in whole blood. In this nonlimiting example, the diagnostic platform comprised a disposable finger stick blood draw kit, handheld reflectance-based meter, the mobile device application, and test strips for Phe detection. As shown in FIG. 2, holders for the mobile device and the test strip may also be included in kits for conducting these methods. The user first opened the mobile device application and turned on the meter. Wireless communication was then established between the mobile device and meter. The user scanned a QR code on the test strip vial to activate calibration curves, data transformation algorithms, endpoint determination, temperature correction algorithms, lot number, lot expiration, timing sequences, event makers and/or quality metrics in the mobile device application for this selected Phe diagnostic assay. The user was then prompted to insert a Phe test strip into the meter. The user then collected and applied a blood sample to the Phe test strip. The diagnostic meter reported reflectance-based data to the mobile phone application and calculated a Phe level for the user. The test strip was then discarded, and the test is repeated as needed.

In this nonlimiting embodiment, the test strips for Phe detection comprised four superimposed layers, 3 of which are membranes. The first layer depicted is a sample spreading layer. The sample spreading layer is capable of distributing or metering the sample's biological cells evenly across the surface of the primary membrane. The spreading layer provides a uniform concentration of cells between the interface of the spreading layer and the primary membrane. The spreading layer can be a mesh material, an isotropically porous membrane (same porosity throughout), or an anisotropic membrane (a gradient in porosity). The spreading layer can be composed of nylon or polyester with a pore size in the range of 10-300 μm. Precise permeability of the spreading layer is critical, as it determines whether or not a homogeneous biological sample will be uniformly distributed across the surface of the primary membrane layer.

The surface of the spreading layer is in direct contact with a primary membrane for uniform transfer of the biological material through a lateral and vertical migration of the biological fluid. The biological fluid flows transversely across the spreading layer before migrating vertically into the primary membrane. In this nonlimiting example, the primary membrane is a blood separation membrane. This primary whole blood separation membrane is referred to herein, as Membrane-1. Membrane-1 contains a non-hemolytic surfactant, hemagglutinating agent, hemoglobin oxidizing agent, polymer, and buffer. Membrane-1 can be composed of one, or a combination of several, material(s) including, but not limited to, glass fiber, nylon, polyester, cellulose, cellulose acetate, nitrocellulose, polycarbonate, polyvinylidene difluoride, polyethersulfone, or polysulfone with a particle retention in the range of 2.0-5.0 μm. Membrane-1 is comprised of hemagglutinating agents, including but not limited to, anti-red blood cell antibodies, chitosan, hexadimethrine bromide, poly-L-lysine, poly-L-lysine hydrobromide, poly-D-lysine, poly-D-lysine hydrobromide, poly-DL-lysine hydrobromide, poly-L-arginine hydrochloride, poly(allylamine hydrochloride), poly(ethylenimine hydrochloride), diethylaminoethyl dextran, poly(n, n-dimethyl-3,5-dimethylene piperidinium chloride), or crude or purified lectins which agglutinate human type 0 erythrocytes efficiently such as those from Phaseolus vulgaris, Maclura pomifera, Ulex europaeus, and Solanum tuberosum. Additionally, the hemagglutinating agents can also be combined with a Neuraminidase, such as those from Clostridium perfringens, Arthrobacter ureafaciens, or Streptococcus pneumonia, to increase the hemagglutination efficiency of any lectins added to the primary blood separation membrane. The hemagglutinating agents can be immobilized together with a polymer, including but not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride).

The PKU test strip further comprises Membrane-2 M2. The plasma and remaining cells from the primary membrane M1 continue migrating vertically downward into the secondary membrane M2. The secondary whole blood separation membrane is referred to herein as Membrane-2. Membrane-2 is in direct contact with Membrane-1. In this nonlimiting example, Membrane-2 is composed of one, or a combination of several, material(s) including, but not limited to, glass fiber, nylon, polyester, cellulose, cellulose acetate, nitrocellulose, polycarbonate, polyvinylidene difluoride, polyethersulfone or polysulfone with a pore size in the range of 0.8-5.0 μm. Membrane-2 contains a non-hemolytic surfactant, polymer, and buffer.

In one non-limiting embodiment, Membrane-2 contains an immobilized preconditioning buffer in the pH range of 6.0 to 8.0. The optimal pH of PheDH can range from 10 to 11.5 depending on the variant. At the pH of optimal activity, the non-specific activity for endogenous L-tyrosine can interfere by as much as 100% in the blood Phe range of 0-6 mg/dL. The preconditioning of the biological fluid allows time for the homogenous mixing of the excipients while also buffering the biological fluid to a suitable pH for the enzymatic determination of Phe which simultaneously decreases the non-specific interaction PheDH has for L-tyrosine. The preconditioning of the biological solution to a lower pH suppresses the utilization of L-tyrosine as a substrate by PheDH. The components on Membrane-2 are immobilized with a polymer. Examples of polymers include, but are not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride).

The test strip of this nonlimiting example further comprises Membrane-3 M3. The buffered fluid containing Phe travels from Membrane-2 to the tertiary membrane M3. The tertiary membrane is referred to herein as “the reagent membrane” or “Membrane-3”. The reagent membrane is visually clean and smooth with submicron-sized pores thus providing excellent optical and reflective properties. Membrane-3 is composed of one, or a combination of several, material(s) including, but not limited to, nylon, cellulose, cellulose acetate, nitrocellulose, polycarbonate, polyethersulfone or polysulfone with a pore size in the range of 0.03-1.2 μm. This reagent membrane provides a uniform end-color in the read zone for precise detection. The reagent membrane contains a phenylalanine dehydrogenase (PheDH), a surfactant, polymer, buffer, an electron mediator, the cofactor β-Nicotinamide adenine dinucleotide or salts thereof, stabilizers, and a tetrazolium salt indicator. In one non-limiting embodiment, the PheDH is from Thermoactinomyces intermedius, Bacillus badius, Sporosarcina ureae, Rhodococcus sp. strain M4, or recombinant derivatives of the latter expressed in Escherichia coli. In one non-limiting embodiment, the electron mediator is a diaphorase, 1-methoxy-5-methylphenazinium methylsulfate (1-methoxy PMS), 1-methoxy-5-ethylphenazinium ethylsulfate (1-methoxy PES), or any combinations thereof. The components on Membrane-3 are immobilized with a polymer including, but not limited to, hydroxypropyl cellulose, hydroxyethyl cellulose, poly(vinyl alcohol), dextran, gelatin, agarose, sodium carboxymethyl cellulose, xanthan gum, polyvinyl pyrrolidone, poly(1-vinylpyrrolidone-co-vinyl acetate), poly(vinyl acetate) or poly(methyl vinyl ether-alt-maleic anhydride). The biological fluid slowly migrates vertically downward onto the reagent membrane. The end-color intensity of the reagent membrane can be measured in percent reflectance units on a handheld meter and converted to mg/dL or micromolar Phe through a preprogrammed curve set, calibrated against a laboratory reference instrument, or as an optical image measuring RGB values, which can then be calibrated against a laboratory reference instrument. The concentration of Phe can be determined by the end-color intensity at a given time or by kinetic rate determination. In one non-limiting embodiment, the reagent membrane is positioned facing a light emitting diode (LED) and photodiode to measure the end-color intensity of the reagent membrane or positioned facing a camera to image the end-color using Red/Green/Blue (RGB) values. In one non-limiting embodiment, the LED and photodiode can detect the end-color of a generated formazan that has a lambda max wavelength in the range of 500 nm and 700 nm for reflectance determination. Quantification of the analyte of interest can be achieved via percent reflectance versus a gold-standard reference instrument. The end-color can also be quantified using a camera to image the end-color intensity of the generated formazan. Quantification by image analysis can be calculated from RGB values.

Phenylalanine dehydrogenase in the presence of an electron mediator, the cofactor β-Nicotinamide adenine dinucleotide (NAD⁺), and a tetrazolium salt indicator, the end-color intensity, or rate of color development, is proportional to the Phe concentration.

Suitable electron mediators or electron transfer agents include, but are not limited to, diaphorase (from Clostridium kluyveri, Bacillus megaterium, Bacillus stearothermophilus, Porcine Heart, or recombinant derivatives of the latter expressed in Escherichia coli), or non-enzymatic electron transfer agents, such as phenazine methosulfate (PMS), phenazine ethosulfate (PES), 1-methoxy-5-methylphenazinium methylsulfate (1-methoxy PMS) or 1-methoxy-5-ethylphenazinium ethylsulfate (1-methoxy PES), can all be used in the reduction of tetrazolium salts. Reaction kinetics and stability are the primary factors for selecting an electron transfer agent or electron mediator. For example, PMS is a good electron mediator because it has relatively fast reaction kinetics with most tetrazolium compounds described herein. PMS, however, is less stable in light than enzyme-based electron mediators such as diaphorase or other PMS derivatives. Diaphorase can be very stable in environmental conditions and, for that reason, is preferred when the cofactor NAD⁺ is used.

PheDH catalyzes the oxidation of Phe and the reduction of NAD⁺ to NADH. The NADH generated is utilized by diaphorase to reduce a tetrazolium salt to its corresponding colored formazan biproduct as in the reaction mechanism below:

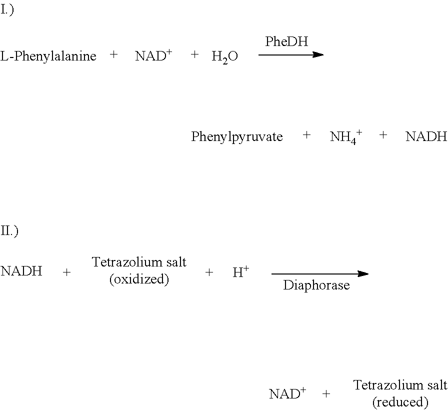

The “normal” range for Phe in the blood is 0-6 mg/dL. This invention demonstrates exceptional performance over the analytical range of 0 to 25 mg/dL (0-1513.4 μM) of Phe. In practice, this nonlimiting embodiment of the present invention determines Phe levels as a point-of-care test. The concentration of Phe in the blood is a critical parameter for neonatal determination of PKU, pre- and post-assessment for those with dietary restrictions, and monitoring after the administration of therapeutic medications.

The volume of blood used in these methods and devices, using a fingerstick whole blood sample, is less than 25 μL. This allows for ease-of-use for the patient.

Three significant contributions of this nonlimiting embodiment of the present invention are the ability to detect low concentration levels of Phe with a high degree of reliability (sensitivity), the ability to discriminate between various concentrations of Phe over the clinically significant range, and immediate access and display of the diagnostic test results on a mobile device. These are achieved in the present invention in part by the coupling of a diaphorase that performs extremely well at the low pH needed for the suppression of L-tyrosine interference, along with a highly sensitive tetrazolium salt indicator that acts as a good substrate for the preferred diaphorase. The harmonization of the diaphorase, at a given pH with a specific tetrazolium salt, provides the necessary sensitivity in the analytical range of 0 to 25 mg/dL Phe, while simultaneously suppressing endogenous L-tyrosine interference typically seen from phenylalanine dehydrogenase.

The following non-limiting example is provided to further illustrate the present invention.

### EXAMPLE

### Example 1: Use of Computer-Implemented Method with POC Device for Measurement of Phenylalanine

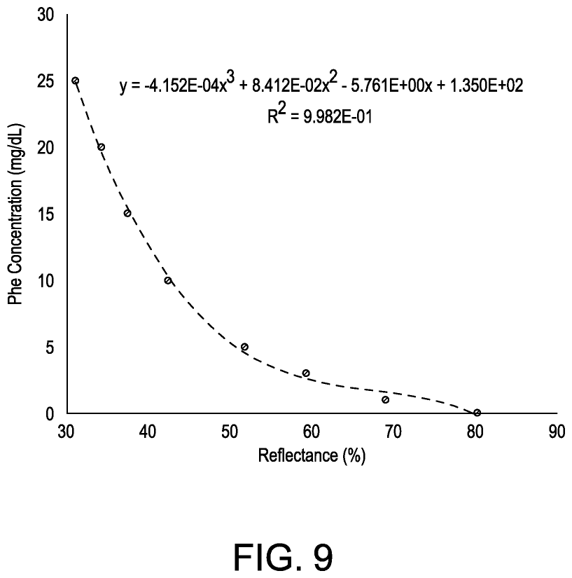

A series of standards from 0-25 mg/dL were prepared by spiking phenylalanine (Phe) into whole blood. Each standard was assayed in triplicate using the PKU Now meter, test strips, and mobile application. Results were averaged to produce a relationship between Phe concentration and reflectance percentage. A cubic equation that best fits this relationship was calculated to obtain the PKU Now calibration equation. Data are depicted in FIG. 9.

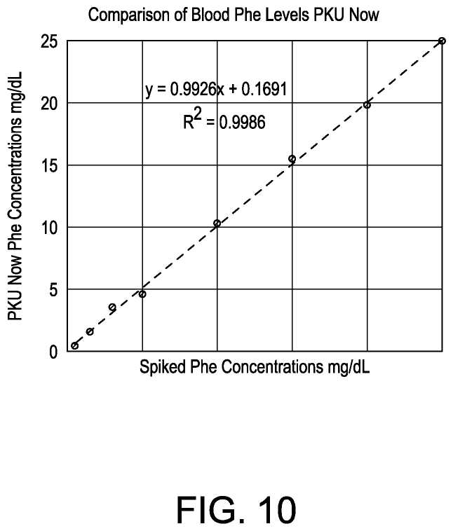

Using the PKU Now calibration equation, percent reflectance was converted to Phe concentration in mg/dL. The predicted value of Phe concentration was compared to the gravimetric measurement of spiked Phe and revealed excellent agreement between measurements. Data are depicted in FIG. 10.

1. A computer-implemented method for capturing and using quantitative and qualitative results from point-of-collection devices for different diagnostic assays on a mobile device application, said method comprising:
connecting a mobile device to a meter of a point-of-collection (POC) diagnostic device;
scanning or imaging a unique code associated with a selected diagnostic assay or test strip lot;
transmitting data from the meter of the POC diagnostic device to an application on the mobile device; and
calculating a diagnostic test result from the transmitted data via the mobile device application.

2. The computer-implemented method of claim 1 wherein the code is a QR code or bar code on a vial of diagnostic test strips.

3. The computer-implemented method of claim 1, wherein the code is associated with calibration curves, transformation, endpoint determination, temperature correction algorithms, lot number, lot expiration, timing sequences, event makers and/or quality metrics in the mobile device application for a selected diagnostic assay.

4. The computer-implemented method of claim 1, wherein the transmitted data comprises photometric, amperometric, conductimetric and/or potentiometric data.

5. The computer-implemented method of claim 1, wherein data from the meter is transmitted to the mobile device via wireless connectivity, near field communication (NFC) or radio frequency identification (RFID).

6. The computer-implemented method of claim 1, wherein the diagnostic test result is calculated via the mobile device application from one or more parameters selected from calibration curves, transformation, endpoint determination, temperature correction algorithms, lot number, lot expiration, timing sequences, event makers and/or quality metrics in the mobile device application.

7. The computer-implemented method of claim 1, wherein the diagnostic test result is qualitative.

8. The computer-implemented method of claim 1, wherein the diagnostic test result is quantitative.

9. The computer-implemented method of claim 1, further comprising a means for inputting patient identification associated with the diagnostic test result.

10. The computer-implemented method of claim 9 wherein the patient information is entered manually into the mobile device application.

11. The computer-implemented method of claim 9 wherein the patient information is associated with a patient specific code imaged by the mobile device.

12. The computer-implemented method of claim 1, wherein the diagnostic test result is immediately accessible and displayed on the mobile device by the mobile device application.

13. The computer-implemented method of claim 1, wherein the diagnostic test result is stored for future viewing and/or dissemination remotely.

14. The computer-implemented method of claim 1, wherein the diagnostic test result is transmitted via the mobile device application to a health care provider or database.

15. The computer-implemented method of claim 14 where the health care provider has access to the database.

16. A device for capturing and using quantitative and qualitative results from POC devices for different diagnostic assays through mobile devices, said device comprising a means for accessing the computer-implemented method of claim 1 via a mobile device and a POC diagnostic device which can connect to said mobile device.

17. The device of claim 16 wherein the POC diagnostic device comprises a meter and test strips for selected diagnostic assays.

18. The device of claim 17 wherein the meter performs photometric, amperometric, conductimetric and/or potentiometric measurements of the test strips for the selected diagnostic assays.

19. A method for calculating a diagnostic test result in a patient, said method comprising:
applying a biological sample obtained from a patient to a diagnostic test strip; and
calculating the diagnostic results indicated by the diagnostic test strip via the computer-implemented method of claim 1.

20. (canceled)

21. A method for calculating a diagnostic test result in a patient, said method comprising:
applying a biological sample obtained from a patient to a diagnostic test strip; and
calculating the diagnostic results indicated by the diagnostic test strip via the device of claim 16.
